(19) United States
(12) Patent Application Publication
(10) Pub. No.: US 2024/0350436 A1
(43) Pub. Date: Oct. 24, 2024

(54) METHODS FOR HAIR GROWTH

(71) Applicant: THE REGENTS OF THE UNIVERSITY OF MICHIGAN, Ann Arbor, MI (US)

(72) Inventors: Rogerio M. Castilho, Ann Arbor, MI (US); Cristiane H. Squarize, Ann Arbor, MI (US)

(21) Appl. No.: 18/579,135

(22) PCT Filed: Jul. 14, 2022
(86) PCT No.: PCT/US22/37139

§ 371 Date: Jan. 12, 2024

Related U.S. Application Data
(60) Provisional application No. 63/221,651, filed on Jul. 14, 2021.

(51) Int. Cl. A61K 31/167; A61K 9/06; A61K 47/38; A61P 17/14

(52) U.S. Cl. CPC A61K 31/167; A61K 9/06; A61K 47/38; A61P 17/14

(57) ABSTRACT

The present disclosure provides for treating hair loss (e.g., inducing or promoting hair growth or regrowth) and promoting or activating hair follicle development (e.g., generating new hair follicles or hair follicle stem cells). More particularly, the present disclosure provides methods comprising administration of PTEN inhibitors (e.g., vanadate derivatives (e.g., bisperoxovanadium salts) to a subject.

### CROSS-REFERENCE TO RELATED APPLICATIONS

This application claims the benefit of U.S. Provisional Application No. 63/221,651, filed Jul. 14, 2021, the content of which is herein incorporated by reference in its entirety.

### STATEMENT REGARDING FEDERALLY-SPONSORED RESEARCH OR DEVELOPMENT

This invention was made with government support under grant number GM120056 awarded by the National Institutes of Health. The government has certain rights in the invention.

### FIELD

The present invention relates to methods for treatment of hair loss or inducing hair growth or regrowth in a subject using PTEN inhibitors. In particular, the present invention relates to methods for treatment of hair loss or inducing hair growth or regrowth in a subject using vanadate derivatives (e.g., bisperoxovanadium salts).

### BACKGROUND

By age 50 approximately 85% of men and 50% of women will experience hair thinning or loss of hair. While hair loss, also known as alopecia, can occur due to a variety of causes including: medical conditions, effect of common cancer treatments, aging, environmental factors, and genetic factors; the two major hair loss disorders are androgenic alopecia and alopecia areata. Current therapies can slow or stop continued hair loss, but very few treatments actually promote the regrowth of hair. Use of minoxidil or finasteride, the only two FDA approved drugs for alopecia, is accompanied with possible severe side effects and, oftentimes, negative results.

### SUMMARY

Provided herein methods of treating hair loss in a subject comprising administering an effective amount of an inhibitor of protein phosphotyrosine phosphatase (PTP) and phosphatase and tensin homolog (PTEN) to the subject. In some embodiments, the PTEN inhibitor comprises β-glycerol phosphate disodium salt pentahydrate, a vanadate derivative, SF1670, thioredoxin-1, indolecarboxylic acid salts, nonenal, or a combination thereof. In some embodiments, the PTEN inhibitor is a vanadate derivative, or a pharmaceutical acceptable salt thereof. In some embodiments, the administering comprises topical administration. In some embodiments, the hair loss is due to androgenic alopecia. In some embodiments, the hair loss is due to alopecia areata.

Also provided are methods for promoting or activating hair follicle development comprising contacting an effective amount of an inhibitor of protein phosphotyrosine phosphatase (PTP) and phosphatase and tensin homolog (PTEN) to a hair follicle or hair follicle stem cell. In some embodiments, the PTEN inhibitor is a vanadate derivative, or a pharmaceutical acceptable salt thereof. In some embodiments, the hair follicles are in vivo. In some embodiments, the contacting comprises topical administration of the vanadate derivative, or pharmaceutical acceptable salt thereof, to a subject. In some embodiments, the hair follicles are in vitro.

In some embodiments, the vanadate derivative, or pharmaceutical acceptable salt thereof, comprises a peroxovanadium compound or a pharmaceutical acceptable salt thereof. In some embodiments, the vanadate derivative, or pharmaceutical acceptable salt thereof, comprises bisperoxovanadate or a pharmaceutical acceptable salt thereof. In some embodiments, the vanadate derivative comprises dipotassium bisperoxo(pyridine-2-carboxyl)oxovanadate (bpV(pic)).

In some embodiments, the PTEN inhibitor or pharmaceutical acceptable salt thereof is provided in the form of a composition. In some embodiments, the composition is a cream, a gel, a lotion, an emulsion, a suspension, an oil, or an aqueous or non-aqueous solution.

In some embodiments, the effective amount of the PTEN inhibitor, or pharmaceutical acceptable salt thereof, is administered daily. In some embodiments, the PTEN inhibitor, or pharmaceutical acceptable salt thereof, is administered for at least 14 days.

Kits comprising vanadate derivatives or compositions thereof as described herein are also provided. In some embodiments, the kit further comprises one or more reagent, shipping and/or packaging containers, an application device, instructions, or a combination thereof.

Other aspects and embodiments of the disclosure will be apparent in light of the following detailed description.

### BRIEF DESCRIPTION OF THE DRAWINGS

FIGS. 1A-1D show hair growth as a result of bpV(pic) treatment in comparison to a vehicle control. FIG. 1A is a schematic of the treatment regime. FIG. 1B is a graph of hair length over the treatment regime showing that bpV(pic) induces hair regrowth with daily treatment. FIGS. 1C and 1D show the effect of hair regrowth in females (FIG. 1C) and males (FIG. 1D) with graphs of hair length on the left and images of treated area on the right.

FIGS. 2A-2C are histological analysis of hair follicles following bpV(pic) treatment in comparison to a vehicle control. FIG. 2A are images showing hair follicle growth. The elongated hair follicles are in the anagen phase which denotes activation of hair growth. FIGS. 2B and 2C is the quantitative analysis of proliferative cells in the hair follicles for the identified timepoints.

FIGS. 3A-3D show the effects of bpV(pic) treatment on hair follicle stem cells. FIG. 3A is a schematic of hair follicle stem cells. FIGS. 3B-3D show increased numbers of hair follicle stem cells as determined by FACS analysis in mice (FIGS. 3B and 3C) and increased stem cell number in human stem cells (FIG. 3D) as a result of bpV(pic) treatment compared to a vehicle control.

FIG. 4 is a graph of hair regrowth following topical application of SF16770.

### DETAILED DESCRIPTION

The disclosed methods are useful for treating hair loss (e.g., inducing or promoting hair growth or regrowth) and promoting or activating hair follicle development (e.g., generating new hair follicles or hair follicle stem cells). The methods comprise administration of a PTEN inhibitor. The methods also comprise administration of a vanadate derivative, or pharmaceutical acceptable salt thereof (e.g., dipotassium bisperoxo(pyridine-2-carboxyl)oxovanadate (bpV(pic)). Treatment with dipotassium bisperoxo(pyridine-2-carboxyl)oxovanadate (bpV(pic)) increased proliferation of skin and hair cells but also increased the amount of hair follicle stem cells. Topical treatment with SF1670 (N-(9,10-Dioxo-9,10-dihydro-phenanthren-2-yl)-2,2-dimethylpropionamide, N-(9,10-Dihydro-9,10-dioxo-2-phenanthrenyl)-2,2-dimethyl-propanamide) resulted in faster regrowth of hair.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “contacting” as used herein refers to bring or put in contact, to be in or come into contact. The term “contact” as used herein refers to a state or condition of touching or of immediate or local proximity. Contacting a composition or agent to a target destination, may occur by any means of administration known to the skilled artisan.

As used herein, the terms “administering,” “providing,” and “introducing,” are used interchangeably herein and refer to the placement of the compounds (e.g., vanadate derivatives) or compositions of the disclosure into a subject by a method or route which results in at least partial localization to a desired site. The compounds or compositions can be administered by any appropriate route which results in delivery to a desired location in the subject.

As used herein, “treat,” “treating” and the like means a slowing, stopping, or reversing of progression of a disease, disorder, condition, or status when provided the compounds or compositions described herein to an appropriate subject. The term also means a reversing of the progression of such a disease, disorder, or condition. As such, “treating” means an application or administration of the methods described herein to a subject, where the subject has a disease or a symptom of a disease, disorder, condition, or status where the purpose is to cure, heal, alleviate, relieve, alter, remedy, ameliorate, improve, or affect the disease or symptoms of the disease, disorder, condition, or status.

A “subject” or “patient” may be human or non-human and may include, for example, animal strains or species used as “model systems” for research purposes, such a mouse model as described herein. The subject may include males or females. Likewise, a subject may include either adults or juveniles (e.g., children). Moreover, a subject may mean any living mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Methods of Treatment

The present disclosure provides methods for treating hair loss (e.g., inducing or promoting hair growth). The hair loss may be located anywhere on the body of the subject. In some embodiments, the location of hair loss is the scalp, eyebrow, or eye lash.

Hair loss, or alopecia, can be caused by a variety of conditions and disorders, including but not limited to: heredity (androgenic alopecia); age (involutional alopecia); hair loss disorders: cancer treatment; childbirth; illness; stress; hair care products and styling treatments; friction or pulling; hormones; infections; medication; sexually transmitted infection; thyroid disease; nutrition; poisons; smoking; menopause; and hair shaft abnormalities.

The hair loss may be due to: androgenic alopecia—hereditary pattern hair loss; involutional alopecia—age related hair thinning; alopecia areata—patchy hair loss, usually as a result of an autoimmune disease; anagen effluvium—rapid hair loss resulting from medical treatment, such as chemotherapy; alopecia universalis—all body hair; trichotillomania—a physiological disorder in which a subject pulls out own hair; telogen effluvium—temporary hair thinning over the scalp due to growth cycle of hair or as a result of a medical event or condition; tinea capitis—fungal infection of the scalp; or cicatricial alopecia—an inflammatory response destroys hair follicles and causes scar tissue to form.

In some embodiments, the hair loss is due to androgenic alopecia. In some embodiments, the hair loss is due to alopecia areata.

The present disclosure also provides methods for promoting or activating hair follicle development. Promoting or activating hair follicle development may result in generating new hair follicles or hair follicle stem cells or may result in shifting the phase of the hair follicles into the anagen growth phase. In some embodiments, the hair follicles or hair follicle stem cells are in vivo. In some embodiments, the hair follicles or hair follicle stem cells are in vitro.

The methods comprise administering to the subject or contacting a hair follicle or hair follicle stem cell with an effective amount of an inhibitor of protein phosphotyrosine phosphatase (PTP) and phosphatase and tensin homolog (PTEN) to the subject. PTEN inhibitors are known in the art and include, for example, β-glycerol phosphate disodium salt pentahydrate, vanadate derivatives, SF1670, thioredoxin-1, indolecarboxylic acid salts, and nonenal.

The methods may comprise contacting an effective amount of a vanadate derivative, or a pharmaceutical acceptable salt thereof, to a hair follicle or hair follicle stem cell or administering to the subject an effective amount of a vanadate derivative, or a pharmaceutical acceptable salt thereof. In some embodiments, the contacting comprises topical administration to a location for hair follicle development in a subject.

As used herein, the term “vanadate derivative” includes any compound comprising a form of vanadate. Vanadate is an anionic coordination complex of vanadium, most often due to oxidation, usually in a +5 oxidation state, or formation of oxoanions of vanadium.

In some embodiments, the vanadate derivative comprises a peroxovanadium compound or a pharmaceutical acceptable salt thereof. Peroxovanadium (pV) compounds contain one or two peroxo anion(s), an oxo anion, and an ancillary ligand in the inner coordination sphere of vanadium. Peroxovanadium (pV) compounds and methods of forming thereof are not in the art. See, for example, International Patent Publication No. WO2001012180 and Shaver et al. Molecular and Cellular Biochemistry 153:5-15, 1995, both incorporated herein by reference in their entirety.

In some embodiments, the vanadate derivative comprises a bisperoxovanadate compound. In some embodiments, the vanadate derivative comprises a monoperoxovandate compound.

The peroxovanadium (pV) compound, or a pharmaceutical acceptable salt thereof, may be selected from the group consisting of: dipotassium bisperoxo(5-hydroxypyridine-2-carboxyl)oxovanadate; potassium bisperoxo(bipyridine)oxovanadate; potassium bisperoxo (1,10-phenanthroline)oxovanadate; dipotassium bisperoxo(pyridine-2-carboxyl)oxovanadate; and bis(N,N-dimethylhydroxamido)hydroxooxovanadate. In select embodiments, the vanadate derivative comprises dipotassium bisperoxo(pyridine-2-carboxyl)oxovanadate (bpV(pic)).

The PTEN inhibitor (e.g., vanadate derivative) may be administered in a pharmaceutical composition which further comprises one or more pharmaceutically acceptable carriers. Pharmaceutically acceptable carriers may include any and all solvents, non-toxic, inert solid, semi-solid or liquid fillers, diluents, or formulation auxiliaries of any type. The choice of excipients or pharmaceutically acceptable carriers will depend on factors including, but not limited to, the particular mode of administration and the effect of the excipient on the stability of vanadate derivative.

In some embodiments, the composition is suitable for topical administration. Topical compositions include: PTEN inhibitor (e.g., a vanadate derivative), or a pharmaceutically acceptable salt thereof, and a carrier. Topical compositions include those in the form of a cream, a gel, a lotion, an emulsion, a suspension, an oil, or an aqueous or non-aqueous solution.

Carriers of topical compositions preferably aid in penetration of the active agent, e.g., a vanadate derivative, into the skin. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier(s) of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl etber, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

Suitable thickeners include cetyl alcohol, stearic acid, carnauba wax, hydroxyethyl cellulose, hydroxypropyl cellulose, alginic acid, guar gum, xanthan gum, beeswax, gelatin, magnesium aluminum silicate, silica, bentonite, acrylic acid polymers, cetyl palmitate, ammonium acryloyldimethyltaurate. The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

Fragrances include essential oils, fragrance oils comprising aromatic compounds and chemicals, absolute extracts, and the like. The amount of fragrance in a topical composition is typically about 0% to about 0.5%, particularly, about 0.001% to about 0.1%.

The topical composition may further comprise suitable pH adjusting additives including HCl or NaOH in amounts sufficient to adjust the pH to acceptable values.

The use of such pharmaceutically acceptable carriers for pharmaceutically active substances is well known in the art. The compositions and methods for their preparation will be readily apparent to those skilled in the art. Techniques and formulations may be found, for example, in Remington's Pharmaceutical Sciences, 19th Edition (Mack Publishing Company, 1995).

The PTEN inhibitor (e.g., vanadate derivative) may be administered by any desired route, including, but not limited to, systemic administration or topical administration.

In some embodiments, the PTEN inhibitor (e.g., vanadate derivative) is administered topically. Compositions that can be applied locally to the skin may be in any form including solids, solutions, oils, creams, ointments, gels, lotions, pastes, foams, shampoos, leave-on and rinse-out hair conditioners, milks, cleansers, moisturizers, sprays, skin patches, and the like. In some embodiments, the vanadate derivative is added to or mixed with existing topical products (e.g., sunscreens, lotions, shampoos, and the like).

The specific dosing may depend upon a variety of factors including the age, body weight, and general health of the subject and severity of hair loss. An “effective amount” is an amount that is delivered to a subject, either in a single administration or as part of a series, which achieves the desirable effect, e.g., stimulation of hair growth.

The effective amount may be from 0.01-μM to 1 mM of the PTEN inhibitor (e.g., vanadate derivative). In some embodiments, the effective amount may between 0.01 and 100 μM; between 0.01 and 10 μM; between 0.01 and 1 μM; 0.01 and 0.1 μM; between 0.1 μM and 1 mM; between 0.1 and 100 μM; between 0.1 and 10 μM; between 0.1 and 1 μM; between 1 μM and 1 mM; between 1 and 100 μM; between 1 and 10 μM; between 10 μM and 1 mM; and between 10 and 100 μM of the PTEN inhibitor. In certain embodiments, the effective amount is about 1 μM, about 5 μM, about 10 μM, about 15 μM, about 20 μM, about 25 μM or about 50 of the PTEN inhibitor.

The frequency of dosing the effective amount can vary, but typically the effective amount is delivered daily, either as a single administration or multiple administrations throughout the day.

In some embodiments, the effective amount is delivered more than once daily, for example every 6-8 hours or 2-4 times daily. In some embodiments, the effective amount is delivered less than once daily, for example, every 2 days, twice weekly, or once weekly. In other embodiments, the patient can be dosed more frequently early in the treatment regimen, with decreasing frequency later in the treatment regimen, e.g., once every day for one to 3 weeks, followed by twice weekly until the end of the treatment period.

The treatment period may continue until hair regrowth is determined to be satisfactory to the subject. In some embodiments, the PTEN inhibitor (e.g., vanadate derivative) is administered for one to four weeks or more. In some embodiments, the PTEN inhibitor is administered for about 1 week, about 2 weeks, about 3 weeks, about 4 weeks, about 5 weeks, about 6 weeks, about 7 weeks, about 8 weeks, about 12 weeks, or about 16 weeks. In some embodiments, the treatment period continues after hair is regrown to maintain growth and decrease potential loss of regrown hair.

The methods disclosed herein can be used in conjunction with a wide range of other therapies. The methods disclosed herein may be used before, concomitantly with, or after hair transplant or hair restoration procedure. The methods disclosed herein may be used after medical treatments associated with hair loss (e.g., chemotherapy, radiation therapy).

The PTEN inhibitors, e.g., vanadate derivative, may be administered alone or in combination with a therapeutically effective amount of at least one additional therapeutic agent. In some embodiments, effective combination therapy is achieved with a single composition or formulation that includes both agents, or with two distinct compositions or formulations, administered at the same or different times using the same or different administration methods, wherein one of the compositions or formulations comprises the PTEN inhibitor. In some embodiments, the at least one additional therapeutic agent comprises an anti-inflammatory agent, an antimicrobial agent, or a corticosteroid.

### Kits

The present disclosure provides kits for treating hair loss (e.g., inducing or promoting hair growth or regrowth) and promoting or activating hair follicle development (e.g., generating new hair follicles or hair follicle stem cells) comprising administering an effective amount of PTEN inhibitor (e.g., vanadate derivatives) to the subject.

The kit may comprise an effective amount of a PTEN inhibitor (e.g., vanadate derivative), or a pharmaceutically acceptable salt or composition thereof.

In some embodiments, the PTEN inhibitor (e.g., vanadate derivative), or a pharmaceutical composition thereof, is provided in individual unit administration forms (e.g., individual ampules or tubes). In some embodiments, the PTEN, or a pharmaceutical composition thereof, is provided as multiple unit dosage forms (e.g., a container with a metering value or applicator to supply individual dosages). In some embodiments, the kits further comprise an applicator or application device (e.g., dropper, syringe, brush, sponge, and the like).

In some embodiments, the kit further comprises instructions for using the components of the kit. The instructions are relevant materials or methodologies pertaining to the kit. The materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

In some embodiments, the instructions are in the forms of software for managing automated delivery. The software may be supplied with the system in any electronic form such as a computer readable device, an internet download, or a web-based portal. The software may control various parameters of the delivery, including for example, dosage amounts and volume.

It is understood that the disclosed kits can be employed in connection with the disclosed methods.

### EXAMPLES

The following are examples of the present invention and are not to be construed as limiting.

### Materials and Methods

Animal model Male and female Balb/cJ mice were housed on 12-h light/dark cycles and had free access to food and water at the University of Michigan vivarium, according to AAALAC guidelines. Mice were randomly assigned to the two experimental groups, which received vehicle or bpV(pic) topical treatment twice a day. The dorsal skin of mice was shaved and monitored for 21 days or longer, as described in the pictures. Hair regrowth was monitored daily. Mice pictures were taken, and dorsal hairs were collected using tweezers. The hair length was measured by digital pachymeter. Skin samples were fixed overnight in 4% PFA and further embedded in paraffin. Unstained slides were used for hematoxylin and eosin (H&E) staining performed to evaluate the skin histomorphology.

Drug administration bpV(pic) (Sigma Aldrich, San Louis, MO, USA) was reconstituted in sterile Milli-Q water. bpV(pic) was also used to prepare a 10 μM bpV(pic) gel using 2% carmellose gel base (vehicle). bpV(pic) or vehicle gel was topically applied to the skin twice a day.

Histology and hair growth phase All hematoxylin and eosin (H&E) stains were performed on paraffin-embedded serial tissue sections (3-5 μm thick) according to standard procedures. The hair follicle cycle was evaluated by morphology and divided into 3 phases. The growth phase (anagen), in which the hair follicle presents an elongated shape and is characterized by high levels of proliferating cells. The regression phase (catagen) is characterized by an involution process mainly driven by apoptosis of the lower half of the HF. Finally, Telogen is the last stage of the hair cycle in which quiescent epithelial cells from the upper half of the HF remain quiescent until receiving new stimulation for growth. H&E images were taken using a QImaging Publisher 5 digital camera attached to a Nikon Eclipse 80i microscope (Nikon, Melville, NY) and visualized with the NIS-Elements program (Nikon, Melville, NY). The images were analyzed using ImageJ (NIH, Bethesda, MD, USA).

Flow cytometry analysis Isolation of skin single-cell suspension. Briefly, the subcutis underlying the dorsal skin was removed, and the epithelial cell layer was trypsinized for 2 hours at 37° C. Epithelial cell suspensions were filtered through 70 μm mesh filters (BD Bioscience, San Jose, CA, USA) to achieve a single cell suspension. Cells were maintained on ice. Immunofluorescence staining for CD34-FITC and CD49f-P.E./Cye5 primary antibodies (BD Pharmingen, San Jose, CA, USA) was performed to detected hair follicle stem cells. Antibodies were incubated with the cells for 30 minutes at 4° C. after cells were incubated with FcR blocking reagent (Miltenyi Biotec, Bergisch Gladbach, Germany). Human epidermal keratinocytes (HEK) (Cell Applications, Inc., San Diego, CA) were cultured in Epi-Vita Growth Medium (Cell Applications, Inc., San Diego, CA) and maintained at 37° C. with 5% of CO₂ in a humidified incubator. Human epidermal keratinocytes (HEK) were cultured in a monolayer following instructions for culture of primary cells and received BpV(pic) or vehicle for 48 hrs. Treated cells were trypsinized and stained for CD44-APC (BD Pharmingen, San Jose, CA, USA) and Aldefluor kit (StemCell Technologies, Durham, NC, USA) according to the manufacturer's instructions to detected aldehyde dehydrogenase (ALDH) enzymatic activity. A total of 10,000 events were collected for each reaction using a BD Accuri™ C6 Plus flow cytometer (BD Biosciences San Jose, CA, USA).

BrdU labeling and cell proliferation BrdU (10 mg/ml) was injected in mice via IP two hours previous to the euthanasia. Tissue was collected and processed. The tissue was placed on slides and deparaffinized in xylene substitute and hydrated in descending grades of ethanol. Antigen retrieval was performed by HCL at 37° C. for 1 hour. Anti-BrdU (1:200, Novus Biologicals, Centennial, CO) was incubated overnight at 4° C., followed by secondary antibody conjugated with Alexa 488 incubation and counterstaining with Hoechst 33342. All slides were mounted using Fluoroshield. For quantification, ten fields were captured at a magnification of x400. The images were analyzed using ImageJ (NIH, Bethesda, MD, USA), and the proliferation was expressed as the number of positive cells per field.

Statistical analysis Statistical analyses were performed by ANOVA analysis of variance test and student test using GraphPad Prism 8 (GraphPad Software, San Diego, CA). Asterisks denote statistical significance (NS—p>0.05; * p≤0.05; ** p≤0.01; *** p≤0.001; **** p≤0.0001).

### Example 2

Hair Growth as a Result of bpV(pic) Treatment

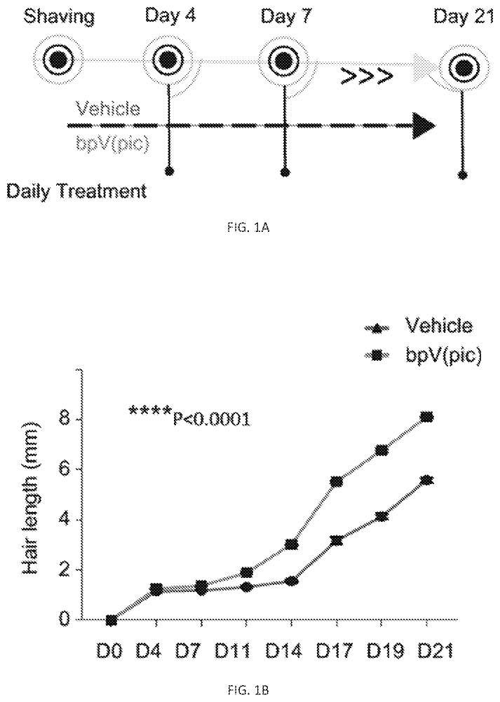

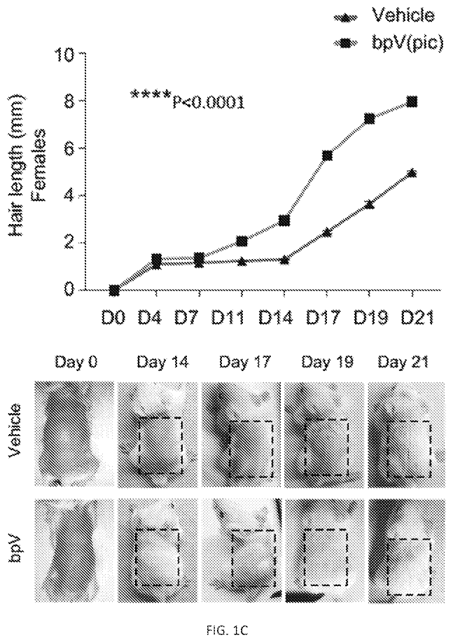

The dorsal skin of male and female mice was shaved and treated with vehicle or bpV (pic) topically, twice a day for 21 days (FIG. 1A). Hair length was monitored throughout treatment. bpV (pic) induced hair regrowth with daily treatment in comparison to the vehicle control over all subject (FIG. 1B) and at similar levels in both female and male mice (FIGS. 1C and 1D, respectively).

### Example 3

bpV(pic) Treatment Effect on Hair Follicles

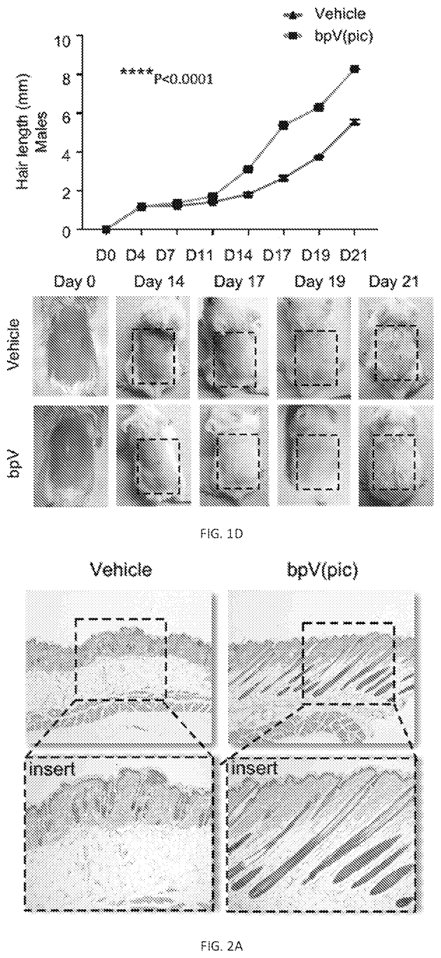

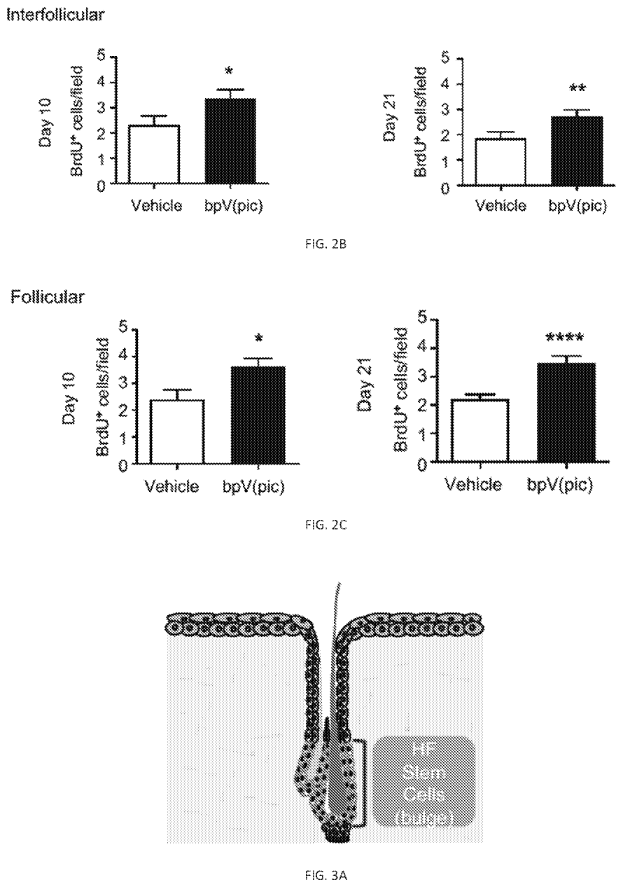

Skin samples were taken from treated and vehicle control mice and stained to evaluate skin histomorphology. Hair follicle cycle was evaluated by morphology and divided into 3 phases: growth phase (anagen); regression phase (catagen); and telogen. The elongated hair follicles for the treated mice (FIG. 2A) are in the anagen phase, denoting activation of hair growth. Proliferative cells in the hair follicle (follicular) and interfollicular epidermis (Interfollicular) were quantified for the identified timepoints. After 10 days proliferative cells increased in the hair follicle and interfollicular epidermis; after 21 days the increase in comparison to vehicle control was even more pronounced (FIGS. 2B and 2C).

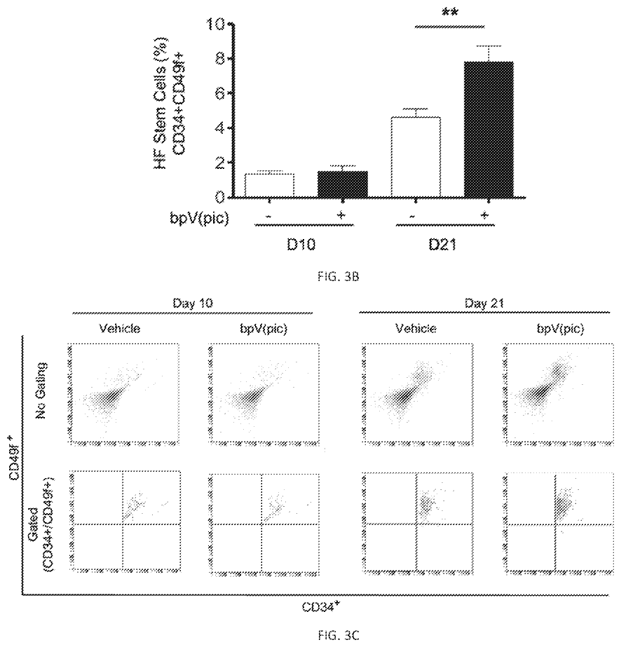

FACS analysis confirmed that bpV(pic) treatment not only increased proliferating cells but also increased the number of hair follicle stem cells in treated mice compared to vehicle control (FIGS. 3B-3C). This result was confirmed using human stem cells (FIG. 3D).

### Example 4

### Hair Growth as a Result of PTEN Inhibitor

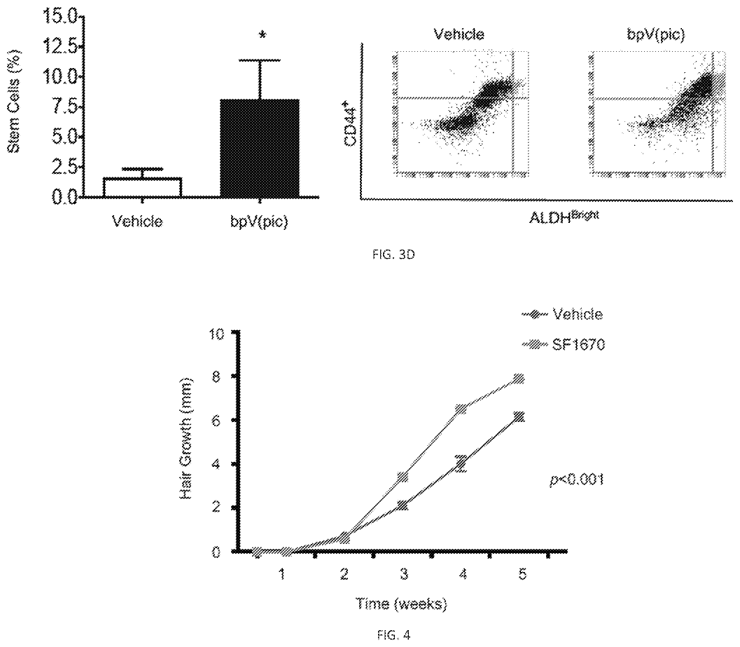

The dorsal skin of male and female mice was shaved and treated with vehicle or SF1670 (N-(9,10-Dioxo-9,10-dihydro-phenanthren-2-yl)-2,2-dimethylpropionamide, N-(9,10-dihydro-9,10-dioxo-2-phenanthrenyl)-2,2-dimethyl-propanamide) topically, twice a day for 5 weeks. Hairs were collected once a week and measured using a millimeter (mm) as the measurement unit. As shown in FIG. 4, SF1670 led to faster hair regrowth in all mice compared to vehicle control).

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method of treating hair loss in a subject comprising administering an effective amount of a phosphatase and tensin homolog (PTEN) inhibitor, or a pharmaceutical acceptable salt thereof, to the subject.

2. The method of claim 1, wherein the hair loss is due to androgenic alopecia.

3. The method of claim 1, wherein hair loss is due to alopecia areata.

4. The method of any of claims 1-3, wherein the PTEN inhibitor comprises β-glycerol phosphate disodium salt pentahydrate, a vanadate derivative, SF1670, thioredoxin-1, indolecarboxylic acid salts, nonenal, or a combination thereof.

5. The method of any of claims 1-4, wherein the PTEN inhibitor comprises a vanadate derivative, or a pharmaceutical acceptable salt thereof.

6. The method of claim 5, wherein the vanadate derivative, or pharmaceutical acceptable salt thereof, comprises a peroxovanadium compound or a pharmaceutical acceptable salt thereof.

7. The method of claim 5 or claim 6, wherein the vanadate derivative, or pharmaceutical acceptable salt thereof, comprises bisperoxovanadate or a pharmaceutical acceptable salt thereof.

8. The method of any of claims 5-7, wherein the vanadate derivative comprises dipotassium bisperoxo (pyridine-2-carboxyl) oxovanadate (bpV(pic)).

9. The method of any of claims 1-8, wherein the PTEN inhibitor or pharmaceutical acceptable salt thereof is provided in the form of a composition.

10. The method of claim 9, wherein the composition is a cream, a gel, a lotion, an emulsion, a suspension, an oil, or an aqueous or non-aqueous solution.

11. The method of any of claims 1-10, wherein the administering comprises topical administration to a location of hair loss.

12. The method of claim 11, wherein the location of hair loss is the scalp, eyebrow, or eye lash of the subject.

13. The method of any of claims 1-12, wherein the effective amount of the PTEN inhibitor or pharmaceutical acceptable salt thereof, is administered daily.

14. The method of any of claims 1-13, wherein the PTEN inhibitor, or pharmaceutical acceptable salt thereof, is administered for at least 14 days.

15. A method for promoting or activating hair follicle development comprising contacting an effective amount of a (PTEN) inhibitor, or a pharmaceutical acceptable salt thereof, to a hair follicle or hair follicle stem cell.

16. The method of claim 15, wherein the PTEN inhibitor comprises B-glycerol phosphate disodium salt pentahydrate, a vanadate derivative, SF1670, thioredoxin-1, indolecarboxylic acid salts, nonenal, or a combination thereof.

17. The method of claim 15 or claim 16, wherein the PTEN inhibitor comprises a vanadate derivative, or a pharmaceutical acceptable salt thereof.

18. The method of claim 16 or claim 17, wherein the vanadate derivative, or pharmaceutical acceptable salt thereof, comprises a peroxovanadium compound or a pharmaceutical acceptable salt thereof.

19. The method of any of claims 16-18, wherein the vanadate derivative, or pharmaceutical acceptable salt thereof, comprises bisperoxovanadate or a pharmaceutical acceptable salt thereof.

20. The method of any of claims 16-19, wherein the vanadate derivative comprises dipotassium bisperoxo (pyridine-2-carboxyl) oxovanadate (bpV(pic)).

21. The method of any of claims 15-20, wherein the PTEN inhibitor, or pharmaceutical acceptable salt thereof, is provided in the form of a composition.

22. The method of claim 21, wherein the composition is a cream, a gel, a lotion, an emulsion, a suspension, an oil, or an aqueous or non-aqueous solution.

23. The method of any of claims 15-22, wherein the hair follicle or hair follicle stem cell is in vitro.

24. The method of any of claims 15-23, wherein the hair follicle or hair follicle stem cell is in vivo.

25. The method of claim 24, wherein the contacting comprises topical administration to a location for hair follicle development in a subject.

26. The method of claim 25, wherein the location of hair follicle development is in the scalp, eye lash, or eyebrow of the subject.

27. The method of claims 25 or claim 26, wherein the effective amount of the PTEN inhibitor, or pharmaceutical acceptable salt thereof, is administered daily.

28. The method of any of claims 25-27, wherein the PTEN inhibitor, or pharmaceutical acceptable salt thereof, is administered for at least 14 days.

29. Use of a PTEN inhibitor, or a pharmaceutical acceptable salt thereof, in the manufacture of a medicament for treating hair loss or inducing or promoting hair growth.

30. Use of a PTEN inhibitor, or a pharmaceutical acceptable salt thereof, in the manufacture of a composition for promoting or activating hair follicle development.

31. The use of claim 29 or claim 30, wherein the PTEN inhibitor comprises β-glycerol phosphate disodium salt pentahydrate, a vanadate derivative, SF1670, thioredoxin-1, indolecarboxylic acid salts, nonenal, or a combination thereof.

32. The use of any of claims 29-31, wherein the PTEN inhibitor comprises a vanadate derivative, or a pharmaceutical acceptable salt thereof,

33. The use of claim 32, wherein the vanadate derivative, or pharmaceutical acceptable salt thereof, comprises a peroxovanadium compound or a pharmaceutical acceptable salt thereof.

34. The use of claim 32 or claim 33, wherein the vanadate derivative, or pharmaceutical acceptable salt thereof, comprises bisperoxovanadate or a pharmaceutical acceptable salt thereof.

35. The use of any of claims 32-34, wherein the vanadate derivative comprises dipotassium bisperoxo(pyridine-2-carboxyl)oxovanadate (bpV(pic)).
